(19) United States
(12) Patent Application Publication
(10) Pub. No.: US 2023/0000460 A1
(43) Pub. Date: Jan. 5, 2023

(54) NON-CONTACT RAPID EYE MOVEMENT (REM) MONITORING

(71) Applicant: Meta Platforms Technologies, LLC, Menlo Park, CA (US)

(72) Inventors: Andre GOLARD, Seattle, WA (US); Sachin TALATHI, Snoqualmie, WA (US); Robert Dale CAVIN, Bellevue, WA (US)

(73) Assignee: Meta Platforms Technologies, LLC, Menlo Park, CA (US)

(21) Appl. No.: 17/856,896

(22) Filed: Jul. 1, 2022

Related U.S. Application Data
(60) Provisional application No. 63/217,554, filed on Jul. 1, 2021.

(51) Int. Cl. A61B 8/08; A61B 8/00; G01S 15/10; G01S 17/10; G01S 7/481; G01S 7/521

(52) U.S. Cl. CPC A61B 8/08; A61B 8/56; A61B 8/4209; A61B 8/4472; A61B 8/4477; G01S 15/10; G01S 17/10; G01S 7/4815; G01S 7/521; A61B 2090/061

(57) ABSTRACT

According to examples, systems, devices, and methods for detecting rapid eye movement (REM) are described. The device may include an array of ultrasound sensors oriented to emit transmit ultrasounds signals in an eyeward direction, wherein the ultrasound sensors are to receive a return signal of the transmit signal reflecting off of a target, and wherein the ultrasound sensors are to output a distance signal representative of a distance to a target, the distance signal generated based on the return signal, and a transceiver to receive the distance signals, wherein the transceiver is to transmit the distance signals from the array of ultrasound sensors to a remote device.

### PRIORITY

This patent application claims priority to U.S. Provisional Patent Application No. 63/217,554, entitled “Non-Contract Rapid Eye Movement (REM) Monitoring,” filed on Jul. 1, 2021, which is hereby incorporated by reference herein in its entirety.

### TECHNICAL FIELD

This patent application relates generally to measurement and monitoring of physiological characteristics, and more specifically, to systems and methods for monitoring non-contact rapid eye movement (REM).

### BACKGROUND

Rapid eye movement (REM) may be an indicator of deep sleep. In some examples, one method for measuring rapid eye movement (REM) sleep may be electro-oculography (EOG). In some instances, electro-oculography (EOG) may be cumbersome as may include contacts being adhered (e.g. glued) to the skin around the eye. In some examples, another method of eye movement tracking may utilize gel electrodes in a mask that can be pressed on to the face rather than being glued to the skin.

### BRIEF DESCRIPTION OF DRAWINGS

Features of the present disclosure are illustrated by way of example and not limited in the following figures, in which like numerals indicate like elements. One skilled in the art will readily recognize from the following that alternative examples of the structures and methods illustrated in the figures can be employed without departing from the principles described herein.

FIG. 1 illustrates an arrangement for implementing electro-oculography (EOG), according to an example.

FIG. 2A illustrates a first view of a mask, according to an example.

FIG. 2B illustrates another (inside) view of a mask, according to an example.

FIG. 3A illustrates sensors in an array for emitting signals, according to an example.

FIG. 3B illustrates a view point of an eye, according to an example.

### DETAILED DESCRIPTION

For simplicity and illustrative purposes, the present application is described by referring mainly to examples thereof. In the following description, numerous specific details are set forth in order to provide a thorough understanding of the present application. It will be readily apparent, however, that the present application may be practiced without limitation to these specific details. In other instances, some methods and structures readily understood by one of ordinary skill in the art have not been described in detail so as not to unnecessarily obscure the present application. As used herein, the terms “a” and “an” are intended to denote at least one of a particular element, the term “includes” means includes but not limited to, the term “including” means including but not limited to, and the term “based on” means based at least in part on.

Embodiments of non-contact ultrasound for rapid eye movement (REM) monitoring are described herein. In the following description, numerous specific details are set forth to provide a thorough understanding of the embodiments. One skilled in the relevant art will recognize, however, that the techniques described herein can be practiced without one or more of the specific details, or with other methods, components, materials, etc. In other instances, well-known structures, materials, or operations are not shown or described in detail to avoid obscuring certain aspects.

Reference throughout this specification to “one embodiment” or “an embodiment” means that a particular feature, structure, or characteristic described in connection with the embodiment is included in at least one embodiment of the present invention. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

Throughout this specification, several terms of art are used. These terms are to take on their ordinary meaning in the art from which they come, unless specifically defined herein or the context of their use would clearly suggest otherwise.

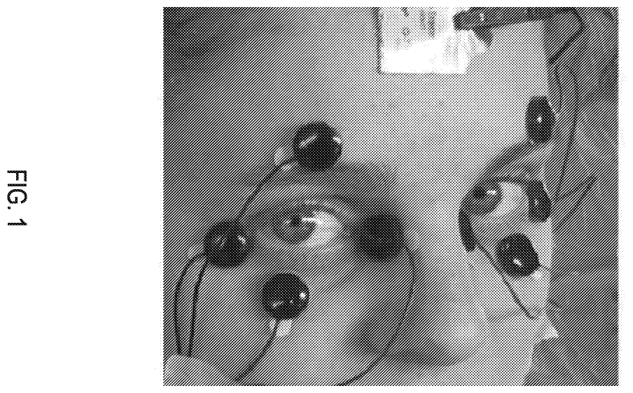

Rapid eye movement (REM) is an indicator of deep sleep. The current method measuring rapid eye movement (REM) sleep is electro-oculography (EOG). Electro-oculography (EOG) is cumbersome as it includes contacts being adhered (e.g. glued) to the skin around the eye, as shown in FIG. 1. Another method of eye movement tracking uses gel electrodes in a mask that can be pressed on to the face rather than being glued to the skin.

Implementations of the disclosure include using non-contact ultrasound sensors or other distance sensors (e.g. LIDAR) to measure eye movement. When the eyes are closed eye rotations (movements) deform the eyelids. Since the eye is non-spherical (the cornea protrudes), movement of the eye under a closed eyelid can be sensed with by sensing the closed eyelid. By using an array of non-contact ultrasound sensors or LIDAR sensors, the movement of the cornea under the eyelid can be measured without having a sensor contact the eye (as required in electro-oculography (EOG)). The ultrasound transducers or other non-contact sensors may be embedded in a sleep mask like device and the distance measurements generated by the array of sensors may be transmitted to a processing unit (e.g. mobile device) for recording and/or analysis.

Using an array of sensors (e.g. ultrasound or LIDAR) for rapid eye movement (REM) monitoring may provide a lightweight, inexpensive, low power, and more comfortable way for detecting periods of rapid eye movement (REM). Conventional airborne ultrasound sensors are used to measure longer distances. For examples, airborne ultrasound sensors are used in the automobile context to measure meters and have centimeter resolution. The ultrasound sensors typically operate at 40-70 kHz. In implementations of the disclosure, the airborne ultrasound sensors operate at in the megahertz (MHz) range (500 kHz to several MHz) and have micron resolution. In some implementations, the ultrasound sensors operate at approximately 1 megahertz (MHz). In some implementations, the ultrasound sensors operate at approximately 1.7 megahertz (MHz). In addition to having better resolution, the increased frequency from conventional ultrasound sensors may also reduce crosstalk between ultrasound sensors in the array.

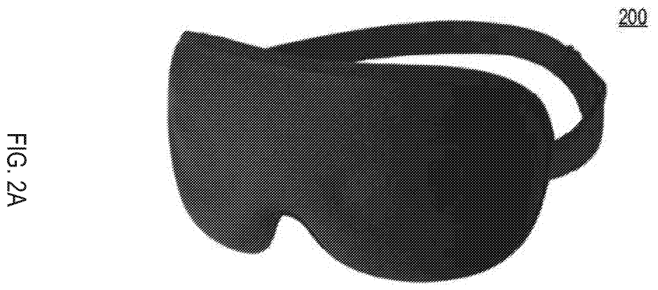

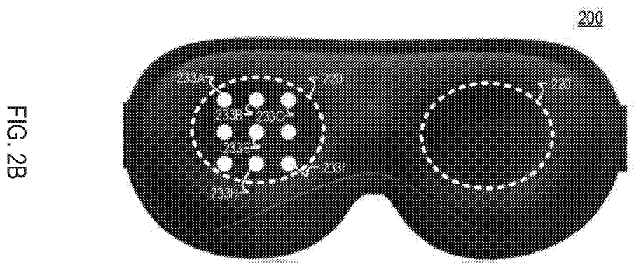

FIG. 2A illustrates an outside of example mask 200. FIG. 2B shows that the inside of mask 200 includes recess cups 220 for the eye to fit into. An array of sensors 233A-2331 are disposed in the recess, in FIG. 2B. The sensors may be airborne ultrasound sensors or other distance sensors. In other implementations, more or fewer sensors 233 may be included in the array. The sensors 233 are oriented to emit transmit signals toward and eye or a user that wears mask 200. The return signal (reflecting from the eye) may be then received by the sensor and a distance to the eyelid (shaped around the eye) may be determined based on the return signal. Batteries, processing logic, communication transceivers, and other electronic components (not illustrated) may also be included in mask 200.

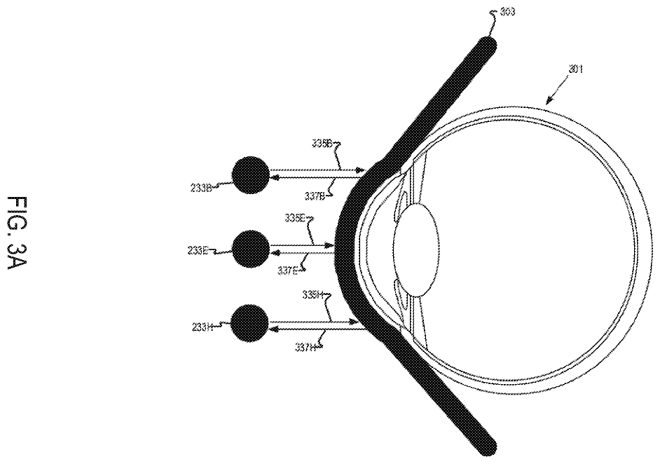

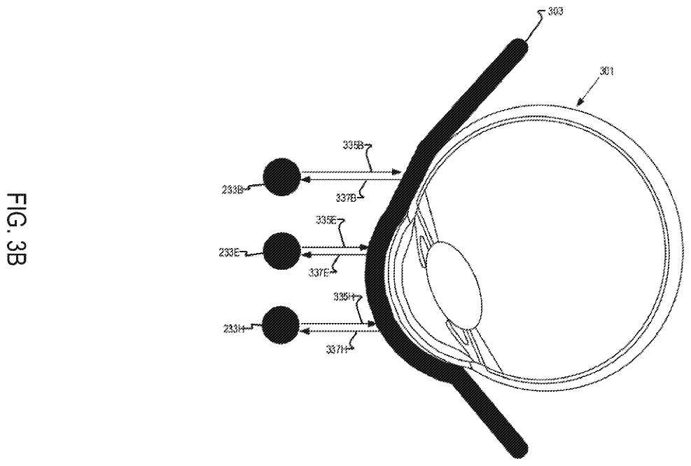

FIG. 3A illustrates sensors 233B, 233E, and 233H in the array emitting transmit signals 335 toward eye 301 and receiving return signals 337 reflected from eyelid 303 that may be shaped around eye 301. Consequently, each sensor 233 measures a distance to eyelid 303. In FIG. 3A, eye 301 may be positioned in a forward-looking direction.

FIG. 3B illustrates eye 301 positioned in a downward-looking direction. In this downward-looking direction, sensor 233E measures a distance to eyelid 303 that is more than the distance measured in the forward-looking direction of FIG. 3A since the center of the cornea is no longer as close to sensor 233E. In FIG. 3B, sensor 233H measures a distance to eyelid 303 that may be less than the distance measured in the forward-looking direction of FIG. 3A. In FIG. 3B, sensor 233B measures a distance to eyelid 303 that may be slightly more than the distance measured in the forward-looking direction of FIG. 3A. Hence, the downward-position of the eye 301 can be determined from the distances to eyelid 303 measured by the array of sensors 233. Of course, other distance measurements would correspond to upward-position and sideways positions of the eye 301. By capturing positions of the eyelid 303 with the array of sensors, REM sleep conditions can be detected.

Embodiments of the invention may include or be implemented in conjunction with an artificial reality system. Artificial reality may be a form of reality that has been adjusted in some manner before presentation to a user, which may include, e.g., a virtual reality (VR), an augmented reality (AR), a mixed reality (MR), a hybrid reality, or some combination and/or derivatives thereof. Artificial reality content may include completely generated content or generated content combined with captured (e.g., real-world) content. The artificial reality content may include video, audio, haptic feedback, or some combination thereof, and any of which may be presented in a single channel or in multiple channels (such as stereo video that produces a three-dimensional effect to the viewer). Additionally, in some embodiments, artificial reality may also be associated with applications, products, accessories, services, or some combination thereof, that are used to, e.g., create content in an artificial reality and/or are otherwise used in (e.g., perform activities in) an artificial reality. The artificial reality system that provides the artificial reality content may be implemented on various platforms, including a head-mounted display (HMD) connected to a host computer system, a standalone HMD, a mobile device or computing system, or any other hardware platform capable of providing artificial reality content to one or more viewers.

The term “processing logic” in this disclosure may include one or more processors, microprocessors, multi-core processors, Application-specific integrated circuits (ASIC), and/or Field Programmable Gate Arrays (FPGAs) to execute operations disclosed herein. In some embodiments, memories (not illustrated) are integrated into the processing logic to store instructions to execute operations and/or store data. Processing logic may also include analog or digital circuitry to perform the operations in accordance with embodiments of the disclosure.

A “memory” or “memories” described in this disclosure may include one or more volatile or non-volatile memory architectures. The “memory” or “memories” may be removable and non-removable media implemented in any method or technology for storage of information such as computer-readable instructions, data structures, program modules, or other data. Example memory technologies may include RAM, ROM, EEPROM, flash memory, CD-ROM, digital versatile disks (DVD), high-definition multimedia/data storage disks, or other optical storage, magnetic cassettes, magnetic tape, magnetic disk storage or other magnetic storage devices, or any other non-transmission medium that can be used to store information for access by a computing device.

Network may include any network or network system such as, but not limited to, the following: a peer-to-peer network; a Local Area Network (LAN); a Wide Area Network (WAN); a public network, such as the Internet; a private network; a cellular network; a wireless network; a wired network; a wireless and wired combination network; and a satellite network.

Communication channels may include or be routed through one or more wired or wireless communication utilizing IEEE 802.11 protocols, BlueTooth, SPI (Serial Peripheral Interface), I2C (Inter-Integrated Circuit), USB (Universal Serial Port), CAN (Controller Area Network), cellular data protocols (e.g. 3G, 4G, LTE, 5G), optical communication networks, Internet Service Providers (ISPs), a peer-to-peer network, a Local Area Network (LAN), a Wide Area Network (WAN), a public network (e.g. “the Internet”), a private network, a satellite network, or otherwise.

A computing device may include a desktop computer, a laptop computer, a tablet, a phablet, a smartphone, a feature phone, a server computer, or otherwise. A server computer may be located remotely in a data center or be stored locally.

The processes explained above are described in terms of computer software and hardware. The techniques described may constitute machine-executable instructions embodied within a tangible or non-transitory machine (e.g., computer) readable storage medium, that when executed by a machine will cause the machine to perform the operations described. Additionally, the processes may be embodied within hardware, such as an application specific integrated circuit (“ASIC”) or otherwise.

A tangible non-transitory machine-readable storage medium includes any mechanism that provides (i.e., stores) information in a form accessible by a machine (e.g., a computer, network device, personal digital assistant, manufacturing tool, any device with a set of one or more processors, etc.). For example, a machine-readable storage medium includes recordable/non-recordable media (e.g., read only memory (ROM), random access memory (RAM), magnetic disk storage media, optical storage media, flash memory devices, etc.).

The above description of illustrated embodiments of the invention, including what is described in the Abstract, is not intended to be exhaustive or to limit the invention to the precise forms disclosed. While specific embodiments of, and examples for, the invention are described herein for illustrative purposes, various modifications are possible within the scope of the invention, as those skilled in the relevant art will recognize.

These modifications can be made to the invention in light of the above detailed description. The terms used in the following claims should not be construed to limit the invention to the specific embodiments disclosed in the specification. Rather, the scope of the invention is to be determined entirely by the following claims, which are to be construed in accordance with established doctrines of claim interpretation.

What has been described and illustrated herein are examples of the disclosure along with some variations. The terms, descriptions, and figures used herein are set forth by way of illustration only and are not meant as limitations. Many variations are possible within the scope of the disclosure, which is intended to be defined by the following claims—and their equivalents—in which all terms are meant in their broadest reasonable sense unless otherwise indicated.

1. A head mounted device comprising:
an array of ultrasound sensors oriented to emit transmit ultrasounds signals in an eyeward direction, wherein the ultrasound sensors are to receive a return signal of the transmit signal reflecting off of a target, and wherein the ultrasound sensors are to output a distance signal representative of a distance to a target, the distance signal generated based on the return signal; and
a transceiver to receive the distance signals, wherein the to transceiver is to transmit the distance signals from the array of ultrasound sensors to a remote device.

2. The head mounted device of claim 1, wherein the array of ultrasound sensors is disposed in recessed cups that are sized to fit an ocular region.

3. A method of detecting rapid eye movement (REM), the method comprising:
emitting signals from an array of sensors in an eyeward direction;
determining distances to an eyelid in response to return signals, wherein the return signals are the transmit signals reflecting off the eyelid; and
determining a rapid eye movement (REM) state of a user in response to the distances.

4. The method of claim 3, wherein the signals are near-infrared light signals and the return signals are also near-infrared light.

5. The method of claim 4, wherein the array of sensors comprises LIDAR sensors.

6. The method of claim 3, wherein the array of sensors is disposed in recessed cups that are sized to fit an ocular region.

7. A head mounted device comprising:
an array of sensors oriented to emit transmit signals in an eyeward direction, wherein the sensors are to receive a return signal of the transmit signal reflecting off of a target, and wherein the sensors are to output a distance signal representative of a distance to a target, the distance signal generated based on the return signal; and
a transceiver to receive the distance signals, wherein the transceiver is to transmit the distance signals from the array of sensors to a remote device.

8. The head mounted device of claim 7, wherein the array of sensors is disposed in recessed cups that are sized to fit an ocular region.

9. The head mounted device of claim 7, wherein the signals are near-infrared light signals and the return signals are also near-infrared light.

10. The head mounted device of claim 7, wherein the array of sensors comprises LIDAR sensors.
